(19) United States
(12) Patent Application Publication
(10) Pub. No.: US 2023/0019775 A1
(43) Pub. Date: Jan. 19, 2023

(54) SPRAY NOZZLE FOR DISPENSING A STRUCTURED COMPOSITION AND A SPRAY PRODUCT COMPRISING THE SAME

(71) Applicant: The Procter & Gamble Company, Cincinnati, OH (US)

(72) Inventors: CODY DYLAN LEWIS, LAWRENCEBURG, IN (US); MATTHEW STEPHEN BAUER, LOVELAND, OH (US); STEVEN ANTHONY HORENZIAK, CINCINNATI, OH (US); MATTHEW LAWRENCE LYNCH, MARIEMONT, OH (US)

(21) Appl. No.: 17/853,966

(22) Filed: Jun. 30, 2022

Related U.S. Application Data
(60) Provisional application No. 63/216,556, filed on Jun. 30, 2021.

(51) Int. Cl. A61L 9/14; B05B 1/34

(52) U.S. Cl. CPC A61L 9/14; B05B 1/3426; A61L 2209/13

(57) ABSTRACT

A spray product is provided. The spray product includes a composition contained within a reservoir. The composition has a yield stress greater than zero and less than 1,000 mPa as determined by the RHEOLOGY TEST METHOD. The spray product includes a valve in composition communication with the reservoir; an actuator in mechanical communication with the valve; a nozzle having an outlet orifice; a swirl chamber in composition communication with the outlet orifice; and a plurality of swirl chamber swirl chamber inlet channels in composition communication with the swirl chamber. The outlet orifice is defined by an outlet orifice diameter and an outlet orifice axial length. A ratio of the outlet orifice diameter to the axial length is from about 1.3 to about 3.5.

### FIELD

The present invention is directed to a spray nozzle for dispensing a structured composition, a spray product comprising the spray nozzle, and a method of spraying a structured composition with the spray nozzle.

### BACKGROUND

Spray products having a trigger are known. Trigger sprayers utilize a handheld container, typically depending from a manual pump or pressurized container. The container may hold any composition desired to be sprayed in a stream, fine droplets, foam or mist. The composition may comprise an air freshener, fabric refresher, hair spray, cleanser, etc.

The characteristics of the spray, e.g. droplet size, spray pattern, and flow rate, are determined by several parameters and operating characteristics of the pump, including nozzle geometry. Further, the rheology of the composition being sprayed also affects the spray characteristics. Specifically, compositions that have a yield stress may spray differently than a water-based composition that is without a yield stress.

There is a need for a spray nozzle design that optimizes the spray characteristics for spraying a composition having a yield stress.

### SUMMARY

Spray products of the present disclosure can exhibit improved spray characteristics for compositions having a yield stress. In one example, a spray product comprises a composition contained within a reservoir, wherein the composition has a yield stress greater than zero and less than 1,000 mPa as determined by the RHEOLOGY TEST METHOD, wherein the spray product further comprises: a valve in composition communication with the reservoir; an actuator in mechanical communication with the valve; a nozzle having an outlet orifice; a swirl chamber in composition communication with the outlet orifice; and a plurality of swirl chamber swirl chamber inlet channels in composition communication with the swirl chamber. The outlet orifice is defined by an outlet orifice diameter and an outlet orifice axial length, wherein a ratio of the outlet orifice diameter to the axial length is from about 1.3 to about 3.5.

In another example, a spray product comprises a composition contained within a reservoir, wherein the composition comprises a plurality of particles and a structurant system comprising xanthan gum a polysaccharide selected from the group consisting of: konjac gum, locust bean gum, tara gum, and combinations thereof, and wherein the composition comprises a plurality of particles, wherein the spray product further comprises: a valve in composition communication with the reservoir; an actuator in mechanical communication with the valve; a nozzle having an outlet orifice; a swirl chamber in composition communication with the outlet orifice; and a plurality of swirl chamber swirl chamber inlet channels in composition communication with the swirl chamber. The outlet orifice is defined by an outlet orifice diameter and an outlet orifice axial length, wherein a ratio of the outlet orifice diameter to the axial length is from about 1.3 to about 3.5.

Additionally, a method of freshening the air, the method comprising the steps of: providing a sprayer and spraying a composition from the sprayer, wherein the composition comprises a plurality of particles and a structurant system comprising xanthan gum a polysaccharide selected from the group consisting of: konjac gum, locust bean gum, tara gum, and combinations thereof, and wherein the composition comprises a plurality of particles. And, the sprayer comprises: a valve in composition communication with the reservoir; an actuator in mechanical communication with the valve; a nozzle having an outlet orifice; a swirl chamber in composition communication with the outlet orifice; and a plurality of swirl chamber swirl chamber inlet channels in composition communication with the swirl chamber. The outlet orifice is defined by an outlet orifice diameter and an outlet orifice axial length, wherein a ratio of the outlet orifice diameter to the axial length is from about 1.3 to about 3.5.

### BRIEF DESCRIPTION OF THE DRAWINGS

FIG. 1 is a side elevation view of a sprayer.

FIG. 2 is a sectional view of an actuator in a forward position of the sprayer of FIG. 1 taken along lines 2-2.

FIG. 3 is a sectional view of an actuator in a rearward position.

FIG. 4 is a fragmentary vertical sectional view of the actuator of FIG. 2, showing the trigger in a rearward position.

FIG. 5 is a perspective view of an actuator, showing the engine housing in phantom.

FIG. 6 is a side elevation view of FIG. 5.

FIG. 7 is an outward-facing plan view of a nozzle.

FIG. 8 is a sectional view of the nozzle of FIG. 7 taken along line B-B.

FIG. 9 is a schematic, fragmentary view of a swirl chamber of a nozzle.

FIG. 10 is a plot of the droplet size versus the spray rate of a nozzle of the present invention.

### DETAILED DESCRIPTION

While the below description describes spray products, including a spray actuator, composition, a housing, trigger, nozzle, and container each having various components, it is to be understood that the spray products is not limited to the construction and arrangement set forth in the following description or illustrated in the drawings. The spray product, including the actuator, housing, trigger, nozzle, pump assembly, container, and composition of the present disclosure are applicable to other configurations or may be practiced or carried out in various ways. For example, the components of the trigger, valve, nozzle having an outlet orifice and swirl chamber, may be used with various pump assemblies for manually-activated trigger sprayers or valve stems of aerosol-type sprayers. Moreover, the trigger and/or pump assembly may be used with various spray actuators for delivering a composition into the air.

The present disclosure relates to a spray product including a spray actuator, a container, and a composition contained with the container. The sprayer of the present disclosure may include an actuator that is optimized to spray a composition having a viscosity in the range of about 1 mPa-s to about 20 mPa-s, as measured according to the RHEOLOGY TEST METHOD disclosed herein. The composition may also have a yield stress of greater than 0 to about 1,000 mPa, as measured according to the RHEOLOGY TEST METHOD disclosed herein. The actuator is optimized to provide a broad spray pattern with small particles and a high flow rate. The composition of the present disclosure may include a plurality of particles and a structurant system to suspend the particles.

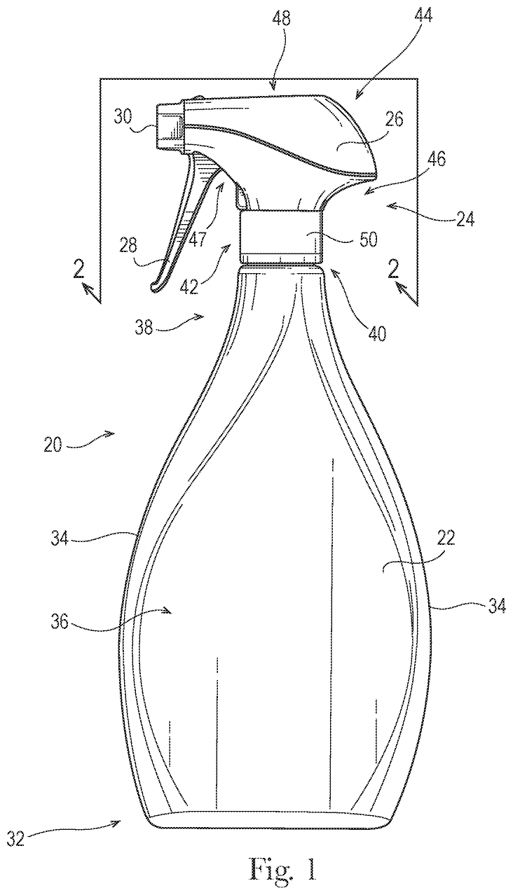

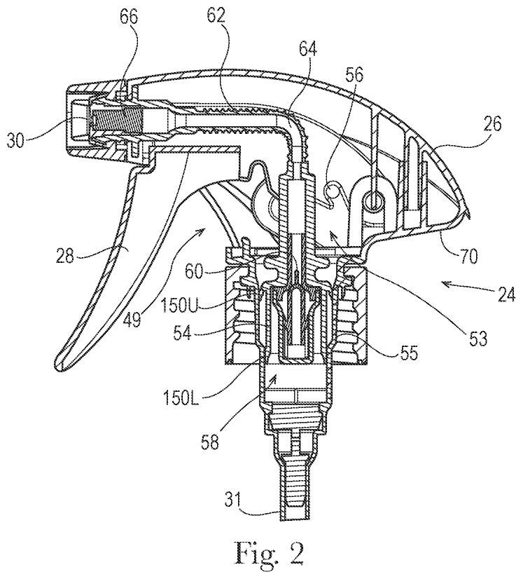

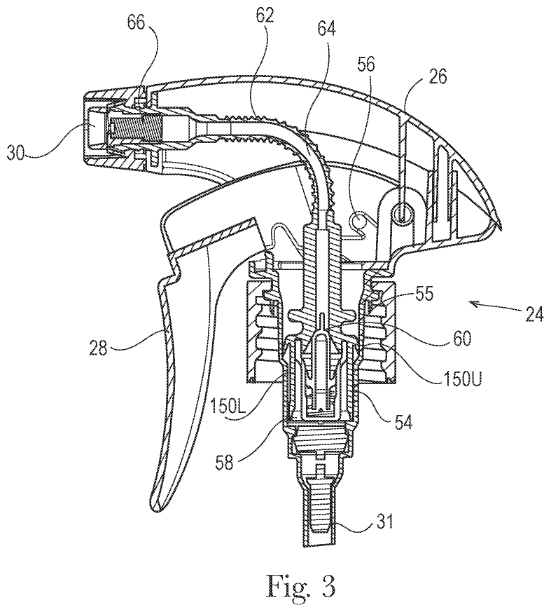

FIGS. 1-6 illustrate non-limiting examples of a spray product 20 of the present disclosure. As shown in FIG. 1, the spray product 20 comprises a container 22 and an actuator 24. The actuator 24 comprises a housing 26, a trigger 28, and a nozzle 30. The spray product 20 may be a non-aerosol manually-actuated trigger sprayer, or any other suitable type of sprayer which can benefit from the features described herein. As noted previously, the actuator 24 or portions thereof may also be used in conjunction with an aerosol type sprayer as well. The spray product 20 and spray actuator 24 may have a longitudinal axis, which is parallel to a portion of the composition flow during dispensing. With reference to FIGS. 2 and 3, the nozzle 30 may be frictionally joined with the housing 26.

With reference to FIG. 1, the container 22 can be any suitable type of container for holding a product to be dispensed by the sprayer. The container 22 may be of any suitable shape. The container 22 has a base 32, sides 34, a lower portion 36, an upper portion 38, and a top 40. The container 22 may be generally cylindrical, but the sides 34 of the container may taper inwardly with a slightly convex curvature on the upper portion 38 of the container, thereby being narrower at its upper portion 38. The base 32, lower portion 36, or sides 34 may be larger (e.g., wider, larger volume, etc.) than the upper portion 38 and/or top 40. The container 22 can have numerous other shapes in different configurations. The container may comprise various materials, including, plastic, metal, glass, the like, and combinations thereof. A single spray actuator 24 can be utilized with various sizes and designs of containers 22.

With reference to FIGS. 1 and 2, the spray actuator 24 may comprise a dip tube 31 that extends from the lower portion 36 of the container 22 to the spray actuator 24. The dip tube 31 may be in composition communication with the composition contained within the container 22 at a first end portion and the spray actuator 24 at an opposite second end portion. The composition contained in the container 22 can be drawn though the dip tube 31, in response to actuation by the trigger 28 or pushed through the dip tube 31 in the case of an aerosol form.

The actuator housing 26 may be configured in various different shapes and sizes. With reference to FIGS. 1 and 2, the actuator housing 26 may have a lower portion 42, an upper portion 44, a waist portion 46, and a top 48. The lower portion 42 can be attached to the container 22, and as shown, can fit on or over the container 22. The waist portion 46 may be disposed between the lower portion 42 and the upper portion 44. The waist portion 46 may be narrower than the widest portions of the upper and/or lower portions 44 and 42, respectively. The waist portion 46 and the lower portion 42 may each be narrower than the widest portions of the upper portion 44. The waist portion 46 provides the actuator 24 with an ergonomic design.

The actuator housing 26 may be configured so that a user can wrap at least their thumb and forefinger around the waist portion 46. In such forms, the actuator housing 26 may also provided with a configuration that permits it to comfortably fit the natural contour of the user's palm, such as in the crease in the user's palm.

The top 48 of the actuator housing 26 may be tilted upward because the sprayer nozzle 30 is oriented so that the composition sprayed from the nozzle 30 will be directed at an angle of greater than 0° and less than 90°. That is, the composition may not be sprayed out parallel to the base 32 (that is, horizontally when the base is placed on a horizontal surface), nor is it sprayed out vertically (straight upward in the direction of the axis of the container). The composition may be sprayed from the nozzle 30 at an angle of greater 0° and less than 90°.

It may be desirable for the composition sprayed from the nozzle 30 to be sprayed horizontally (0°) or vertically (90°). In still other situations, such as in the case of an ironing aid, it may be desirable for the composition sprayed from the nozzle 30 to be directed downward toward a surface (at an angle of between 0° and −90°). It is appreciated, however, that spray patterns are typically in the form of dispersions, and the spray emitted from a nozzle will form a dispersed spray pattern angle when viewed from the side. The angles of spray referred to herein are the central axis that bisects such a spray pattern. It is understood that portions of the spray pattern will typically be distributed on either side of this central axis. With reference to FIGS. 1 and 2, the actuator 24 can be removably affixed to the container 22 in any manner known in the art for removably affixing an article to a container, including but not limited to by screw threads, bayonet fitments, and by a snap fit. The actuator 24 can be permanently affixed to the container 22, or the actuator 24 can be removably affixed to the container 22. Additionally, the actuator housing 26 may include an opening 47 for the trigger 28 to extend therethrough.

Referring to FIGS. 1-6, the spray actuator 24 may be configured as a manually-activated trigger spray actuator or as an aerosol-based trigger spray actuator. A manually-activated spray actuator 24 comprises a pump assembly 53. Manual actuation of the trigger 28 through its stroke causes corresponding vertical movement of a piston 54 of the pump assembly 53. Vertical movement of the piston 54 pumps the composition from the container 22 through a flow path and out the nozzle 30. The piston 54 may move in a reciprocating motion within a pump body 55. The spray product 20 may utilize an articulating, top-pivoting trigger 28.

In contrast, in an aerosol-based system, depression of the trigger opens a valve which allows dispensing of the composition which is under pressure. The composition can be pressurized via known propellants which can be hydrocarbon based, compressed air based or combinations thereof. Hydrocarbon and compressed air propellants are well known in the art.

In either a manually operated trigger sprayer or an aerosol sprayer, a return spring 56 provides bias to force the trigger 28 away from the container 22 and to the forward position (“forward motion”) at the end of the stroke. The return spring(s) 56 may be configured as two curved parallel springs 56. The return springs 56 may be connected at each end and may be disposed outside the piston 54/composition chamber 58. The vertically upwards flow path for the composition may be disposed between the return springs 56. In a manually operated trigger sprayer, squeezing of the trigger 28 toward the container (“rearward motion”) creates hydraulic pressure in a composition chamber 58, causing the composition to be dispensed. Forward motion of the trigger 28 creates a vacuum, drawing the composition from the container 22 to refill the composition chamber 58.

Referring to FIG. 3, once the composition chamber 58 has been primed, rearward motion of the trigger 28 is converted to downward motion of the piston 54 within pump body 55. Downward motion of the piston 54 pressurizes the composition chamber 58. Resistive forces within the system are overcome once the pressure in the composition chamber 58 reaches a predetermined level, causing the valve 60 to open and the composition to flow through a conduit 62 and out the nozzle 30. The return spring 56 automatically alternates the trigger 28 to the forward rest position and the composition chamber 58 is refilled with composition.

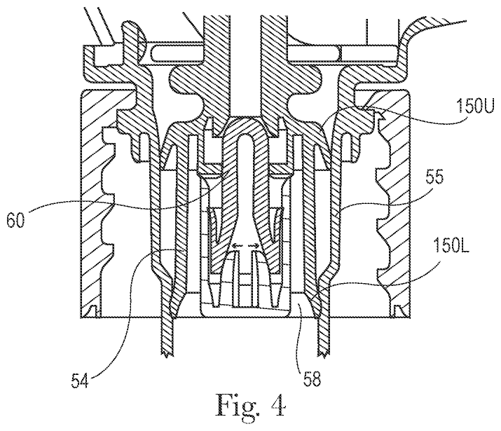

Referring to FIG. 4, and examining the pump assembly 53 in more detail, the pump body 55 may have a stepped configuration and may house the reciprocating piston 54. The pump body 55 may be captured by a screw closure 50 of the lower portion 52 of the actuator 24. The screw closure 50 may be opened to access and replenish the composition in the container 22, as desired. While a screw closure 50 is shown in FIG. 4, it is to be appreciated that the closure may be configured as a different type of closure, such as a bayonet or snap fit.

With reference to FIGS. 2-4, the reciprocating piston 54 may have an upper seal 150U and a lower seal 150L, both of which fit within the body 48. The valve 60 disposed within the piston 54 may have vertical movement thereof resisted by a spring (not shown). As force from the trigger 28 motion increases the force applied to the piston 54 the valve 55 may move downwardly as the composition is pressurized in the chamber 44 to be later dispensed. The conduit 62 may be configured in various ways. For example, with reference to FIG. 2, the conduit 62 may be flexible and bent at approximately 90 degrees. The flexible conduit 62 bends at the elbow 64 in response to movement of the trigger 28/crank rocker, slightly increasing the angle at the elbow 65. The portion of the conduit 62 downstream of the elbow 64 bend terminates at a post 66. The post 66 is inserted into the nozzle 30, up to the shoulder of the post 66. The post may have two longitudinally opposed ends, an upstream end into which the aforementioned bent conduit 62 is fitted and a downstream end which fits into the nozzle 30.

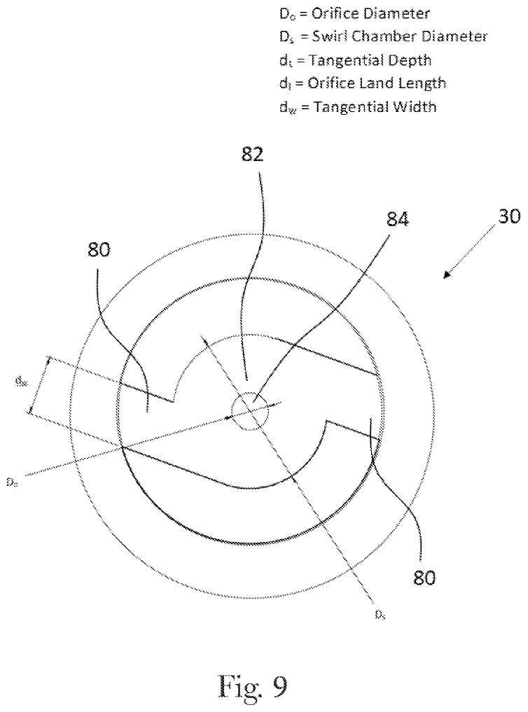

With reference to FIGS. 2 and 7-9, the composition flows around the post 66 and into a plurality of swirl chamber inlet channels 80. The composition flowing from the conduit 62, through the swirl chamber inlet channels 80, which directs the composition into a swirl chamber 82. The swirl chamber inlet channels 80 and swirl chamber 82 impart a tangential rotation to the composition before the composition reaches a nozzle outlet orifice 84 of the nozzle 30. The swirl chamber inlet channels, swirl chamber, and nozzle 30 are stationary. The inlet channels are angled such that the composition enters the swirl chamber at an acute angle to the radius of the swirl chamber.

With reference to FIGS. 2 and 7-8, the swirl chamber inlet channels 80 are defined by the juxtaposition of the outer surface of the post 66 and the inner surface of the nozzle 30. The swirl chamber inlet channels 80 may either be disposed on the post 66, or the swirl chamber inlet channels 80 may be disposed in the nozzle 30. If the swirl chamber inlet channels 80 are disposed on the post 66, the post 66 may have two or more longitudinally oriented slots equally circumferentially spaced around the downstream portion thereof. The post 66 may have a length of about 11 mm and a stepped diameter of about 4-5 mm. The sprayer may include 2, 3, 4, 5, 6, 7, or 8 swirl chamber inlet channels.

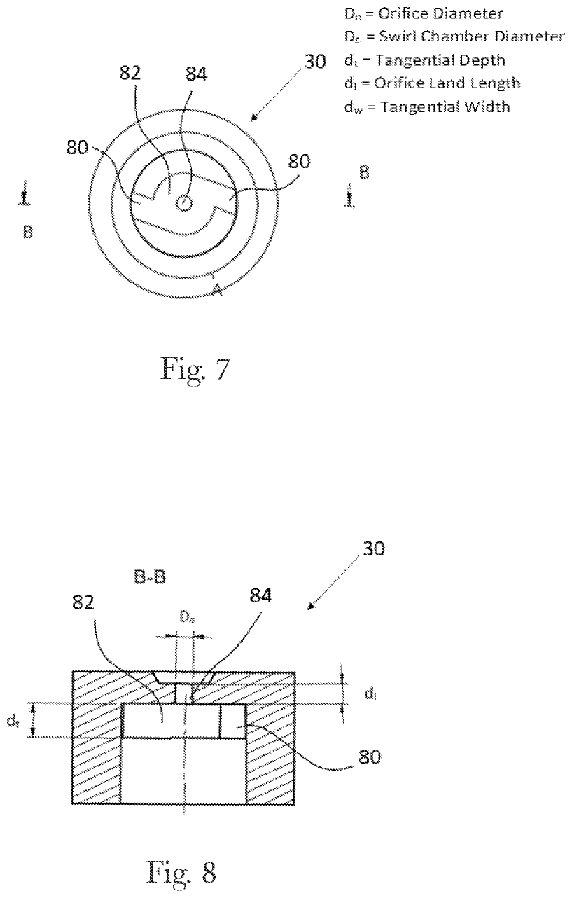

The swirl chamber 82 may either be disposed on the end of the post 66, or the swirl chamber 82 may be disposed at the inner surface of the nozzle 30. FIGS. 2 and 3 illustrate a non-limiting actuator having the swirl chamber 82 and swirl chamber inlet channels 80 disposed on the post 66. FIGS. 7 and 8 illustrate non-limiting nozzles having the swirl chamber 82 and swirl chamber inlet channels 80 disposed on the nozzle.

With reference to FIGS. 2, 3, and 7-9, upon exiting the swirl chamber 82, the composition passes through the nozzle outlet orifice 84 of the nozzle 30 for dispensing into the atmosphere or onto a target surface. The nozzle outlet orifice 84 may be defined by an outlet orifice diameter Do, which is the inner diameter of the outlet orifice, and an outlet orifice axial length di, which is the axial length of the outlet orifice. The nozzle outlet orifice 84 may have an outlet orifice diameter Do and may be radiused on the outside face. The outlet orifice may have an outlet orifice axial length di. A ratio of the outlet orifice diameter Do to the outlet orifice axial length di may be in the range of about 1.3 to about 3.5 or about 1.5 to about 3.2, specifically reciting all values within these ranges and any ranges created thereby. The composition is dispensed from the nozzle 30 in a predetermined spray pattern, which may vary according with the stroke speed, stroke length, etc. of the trigger 28 operation. Optionally, provision may be made for adjusting the spray pattern.

With reference to FIGS. 7-9, the swirl chamber 82 may be defined by a swirl chamber diameter Dₛ. The swirl chamber diameter Dₛ is the transverse internal diameter of the recess that forms the swirl chamber 82. The swirl chamber inlet channels 80 may be defined by a tangential depth dₜ and a tangential width d_{w}. The entire pump assembly 53 may be encased in the housing 70. There may be no direct opening from the pump assembly 53 to the outside of the housing 70, except for the nozzle 30.

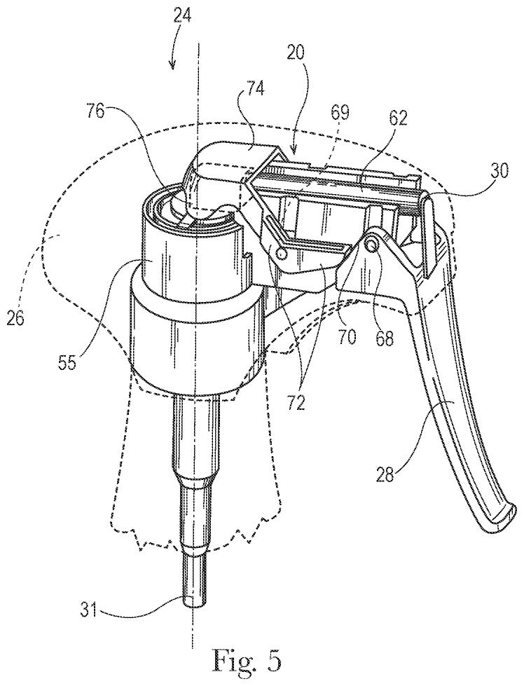

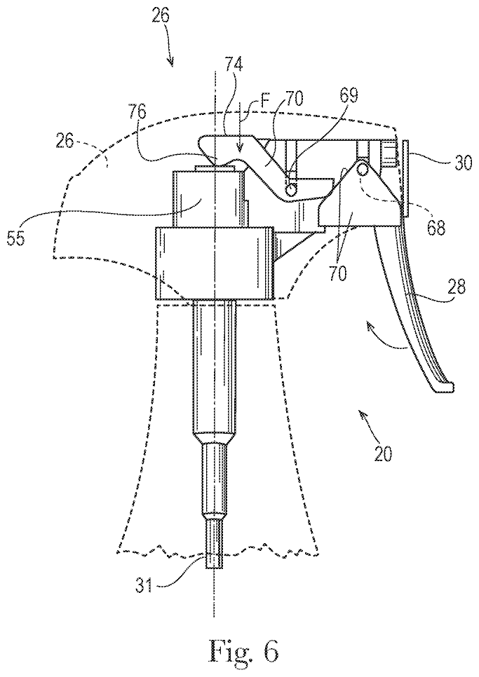

Referring to FIGS. 5-6, the trigger 28 may be configured to provide travel which is more perpendicularly/radially oriented relative to the longitudinal axis than the geometry shown in FIGS. 2-3. This travel orientation may be accomplished by providing mounting trunnions 68 disposed near the uppermost portion of the trigger 28. A rearward-facing protrusion 70 on the trigger 28 may pivot upwardly against a rocker arm 72 of an articulable crank rocker 74. The rocker arm 72 is mounted on two trunnions 69. The opposite end 76 of the crank rocker 74 articulates downwardly, to provide a force F aligned with or coincident the longitudinal axis. This force F displaces the piston 54 in the downward direction, pressurizing composition in the composition chamber 58. Referring back to FIG. 4, composition in the lower portion of composition chamber 58 is displaced by the piston 54, flows upwardly through the annular portion of composition chamber 58, past valve 60 and into conduit 62.

In a manually-actuated trigger sprayer, the pump assembly 53 may be configured as a pre-compression pump assembly as known to one of ordinary skill in the art.

### Composition

The spray product may be used with various compositions. The viscosity of the composition may be in the range of about 1 mPa-s to about 20 mPa-s, about 1 mPa-s to about 15 mPa-s, about 1 mPa-s to about 10 mPa-s, or about 1 mPa-s to about 5 mPa-s, specifically reciting all values within these ranges or any ranges created thereby, as measured according to the RHEOLOGY TEST METHOD disclosed herein.

The yield stress of the composition may be in the range of greater than 0 to about 1,000 mPa, greater than 0 to about 500 mPa, greater than 0 to about 300 mPa, greater than 0 to about 100 mPa, or greater than 0 to about 50 mPa, specifically reciting all values within these ranges or any ranges created thereby, as measured according to the RHEOLOGY TEST METHOD disclosed herein.

Compositions of the present disclosure may include a plurality of particles and a structurant system to suspend the particles.

The composition may have an ionic strength of less than about 0.02 mol/L. Ionic strength is measured according to the following formula:

$$I = \frac{1}{2} {\sum }_{i = 1}^{n} {c}_{i} {z}_{i}^{2}$$

where cᵢ is equal to the molar concentration of ions, i, and had the units mol/L, and
where zᵢ² is equal to the charge number of the ion. See Basic Physical Chemistry, Walter J. Moore, p. 370 (1983).

### Particles

The composition may include a plurality of particles. As used herein, a “particle” may include solid, semi-solid, or liquid droplets. The particle may take various different forms. The particles may be 100 wt. % solid or may be hollow. The particle may include, for example, mesoporous particles, activated carbon, zeolites, benefit agent delivery particle, wax, hydrogel, and/or ground nutwall walls.

The plurality of particles may have an average longest projected dimension in the range of about 0.1 microns to about 500 microns, alternatively about 1 microns to about 100 microns, alternatively about 5 microns to about 50 microns, alternatively less than 100 microns, specifically reciting all values within these ranges and any ranges created thereby. The longest projected dimension of any single particle within the plurality of particles is taken as the length of the longest linear dimension that can be inscribed entirely within the outer perimeter of the single particle. The average longest projected dimension of the plurality of particles may be taken as the average of the longest linear dimension that can be inscribed entirely within the single particle, across all the particles within the plurality of particles. It would be appreciated by one of ordinary skill in the art that this average may also be reflected by taking the average across a statistically relevant sample of particles from the plurality of particles.

As discussed above, the composition may include a particle in the form of a benefit agent delivery particle. The benefit agent delivery particle may include a wall material that encapsulates a benefit agent. Benefit agent may be referred herein as a “benefit agent” or an “encapsulated benefit agent”. The benefit agent may be selected from the group consisting of: a perfume mixture, an insect repellent, a malodor counteractant, and combinations thereof. In one aspect, the perfume delivery technology may comprise benefit agent delivery particles formed by at least partially surrounding a benefit agent with a wall material. The benefit agent may include materials selected from the group consisting of perfume raw materials such as 3-(4-t-butylphenyl)-2-methyl propanal, 3-(4-t-butylphenyl)-propanal, 3-(4-isopropylphenyl)-2-methylpropanal, 3-(3,4-methylenedioxyphenyl)-2-methylpropanal, and 2,6-dimethyl-5-heptenal, alpha-damascone, beta-damascone, gamma-damascone, beta-damascenone, 6,7-dihydro-1,1,2,3,3-pentamethyl-4(5H)-indanone, methyl-7,3-dihydro-2H-1,5-benzodioxepine-3-one, 2-[2-(4-methyl-3-cyclohexenyl-1-yl)propyl]cyclopentan-2-one, 2-sec-butylcyclohexanone, and beta-dihydro ionone, linalool, ethyllinalool, tetrahydrolinalool, and dihydromyrcenol; silicone oils, waxes such as polyethylene waxes; essential oils such as fish oils, jasmine, camphor, lavender; skin coolants such as menthol, methyl lactate; vitamins such as Vitamin A and E; sunscreens; glycerine; catalysts such as manganese catalysts or bleach catalysts; bleach particles such as perborates; silicon dioxide particles; antiperspirant actives; cationic polymers and mixtures thereof. Suitable benefit agents can be obtained from Givaudan Corp. of Mount Olive, N.J., USA, International Flavors & Fragrances Corp. of South Brunswick, N.J., USA, or Firmenich Company of Geneva, Switzerland. As used herein, a “perfume raw material” refers to one or more of the following ingredients: fragrant essential oils; aroma compounds; pro-perfumes; materials supplied with the fragrant essential oils, aroma compounds, and/or pro-perfumes, including stabilizers, diluents, processing agents, and contaminants; and any material that commonly accompanies fragrant essential oils, aroma compounds, and/or pro-perfumes.

The wall material of the benefit agent delivery particle may comprise: melamine, polyacrylamide, silicones, silica, polystyrene, polyurea, polyurethanes, polyacrylate based materials, polyacrylate esters based materials, gelatin, styrene malic anhydride, polyamides, aromatic alcohols, polyvinyl alcohol and mixtures thereof. The melamine wall material may comprise melamine crosslinked with formaldehyde, melamine-dimethoxyethanol crosslinked with formaldehyde, and mixtures thereof. The polystyrene wall material may comprise polyestyrene cross-linked with divinylbenzene. The polyurea wall material may comprise urea crosslinked with formaldehyde, urea crosslinked with gluteraldehyde, polyisocyanate reacted with a polyamine, a polyamine reacted with an aldehyde and mixtures thereof. The polyacrylate based wall materials may comprise polyacrylate formed from methylmethacrylate/dimethylaminomethyl methacrylate, polyacrylate formed from amine acrylate and/or methacrylate and strong acid, polyacrylate formed from carboxylic acid acrylate and/or methacrylate monomer and strong base, polyacrylate formed from an amine acrylate and/or methacrylate monomer and a carboxylic acid acrylate and/or carboxylic acid methacrylate monomer, and mixtures thereof.

The polyacrylate ester-based wall materials may comprise polyacrylate esters formed by alkyl and/or glycidyl esters of acrylic acid and/or methacrylic acid, acrylic acid esters and/or methacrylic acid esters which carry hydroxyl and/or carboxy groups, and allylgluconamide, and mixtures thereof.

The aromatic alcohol-based wall material may comprise aryloxyalkanols, arylalkanols and oligoalkanolarylethers. It may also comprise aromatic compounds with at least one free hydroxyl-group, especially preferred at least two free hydroxy groups that are directly aromatically coupled, wherein it is especially preferred if at least two free hydroxy-groups are coupled directly to an aromatic ring, and more especially preferred, positioned relative to each other in meta position. It is preferred that the aromatic alcohols are selected from phenols, cresoles (o-, m-, and p-cresol), naphthols (alpha and beta-naphthol) and thymol, as well as ethylphenols, propylphenols, fluorphenols and methoxyphenols.

The polyurea based wall material may comprise a polyisocyanate. The polyisocyanate may be an aromatic polyisocyanate containing a phenyl, a toluoyl, a xylyl, a naphthyl or a diphenyl moiety (e.g., a polyisocyanurate of toluene diisocyanate, a trimethylol propane-adduct of toluene diisocyanate or a trimethylol propane-adduct of xylylene diisocyanate), an aliphatic polyisocyanate (e.g., a trimer of hexamethylene diisocyanate, a trimer of isophorone diisocyanate and a biuret of hexamethylene diisocyanate), or a mixture thereof (e.g., a mixture of a biuret of hexamethylene diisocyanate and a trimethylol propane-adduct of xylylene diisocyanate). In still other embodiments, the polyisocyante may be cross-linked, the cross-linking agent being a polyamine (e.g., diethylenetriamine, bis(3-aminopropyl)amine, bis(hexanethylene)triamine, tris(2-aminoethyl)amine, triethylenetetramine, N,N′-bis(3-aminopropyl)-1,3-propanediamine, tetraethylenepentamine, pentaethylenehexamine, branched polyethylenimine, chitosan, nisin, gelatin, 1,3-diaminoguanidine monohydrochloride, 1,1-dimethylbiguanide hydrochloride, or guanidine carbonate).

The polyvinyl alcohol based wall material may comprise a crosslinked, hydrophobically modified polyvinyl alcohol, which comprises a crosslinking agent comprising i) a first dextran aldehyde having a molecular weight of from 2,000 to 50,000 Da; and ii) a second dextran aldehyde having a molecular weight of from greater than 50,000 to 2,000,000 Da.

The wall can include a first wall component and a second wall component, wherein the second wall component is surrounding the first wall component. The first wall component can include a condensed layer and a nanoparticle layer, wherein the condensed layer is disposed between the core and the nanoparticle layer. The condensed layer can include a condensation product of a precursor. The second wall component can include an inorganic coating, wherein the inorganic coating surrounds the nanoparticle layer. The precursor comprises at least one compound of Formula (I): (M^{v}O_{z}Yₙ)_{w} (Formula I), where M is one or more of silicon, titanium and aluminum, v is the valence number of M and is 3 or 4, z is from 0.5 to 1.6 or 0.5 to 1.5, each Y is independently selected from —OH, —OR², halo,

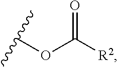

—NH₂, —NHR², —N(R²)₂, and

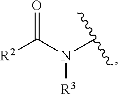

wherein R² is a C₁ to C₂₀ alkyl, C₁ to C₂₀ alkylene, C₆ to C₂₂ aryl, or a 5-12 membered heteroaryl comprising from 1 to 3 ring heteroatoms selected from O, N, and S, R³ is a H, C₁ to C₂₀ alkyl, C₁ to C₂₀ alkylene, C₆ to C₂₂ aryl, or a 5-12 membered heteroaryl comprising from 1 to 3 ring heteroatoms selected from O, N, and S, n is from 0.7 to (v−1), and w is from 2 to 2000.

The perfume benefit agent delivery particle may be coated with a deposition aid, a cationic polymer, a non-ionic polymer, an anionic polymer, or mixtures thereof. Suitable polymers may be selected from the group consisting of: polyvinylformaldehyde, partially hydroxylated polyvinylformaldehyde, polyvinylamine, polyethyleneimine, ethoxylated polyethyleneimine, polyvinylalcohol, polyacrylates, and combinations thereof. The composition may include one or more types of benefit agent delivery particles, for examples two benefit agent delivery particles types, wherein one of the first or second benefit agent delivery particles (a) has a wall made of a different wall material than the other; (b) has a wall that includes a different amount of wall material or monomer than the other; or (c) contains a different amount perfume oil ingredient than the other; (d) contains a different perfume oil; (e) has a wall that is cured at a different temperature; (0 contains a perfume oil having a different c Log P value; (g) contains a perfume oil having a different volatility; (h) contains a perfume oil having a different boiling point; (i) has a wall made with a different weight ratio of wall materials; (j) has a wall that is cured for different cure time; and (k) has a wall that is heated at a different rate.

The perfume benefit agent delivery particle can have a wall material comprising a polymer of acrylic acid or derivatives thereof and a benefit agent comprising a perfume mixture.

The composition may comprise any amounts of particles. With regard to benefit agent delivery particles, the composition may contain from about 0.001 wt. % to about 2.0 wt. %, by weight of composition, of benefit agent contained with the wall material of the benefit agent delivery particle. Or, the composition may contain from about 0.01 wt. % to about 1.0 wt. %, or from about 0.05 wt. % to about 0.5 wt. %, by weight of composition, of benefit agent contained within the wall material of the benefit agent delivery particle.

With regard to unencapsulated perfume, the composition may include from about 0.001 wt. % to about 2.0 wt. %, or from about 0.01 wt. % to about 1.0 wt. %, or from about 0.05 wt. % to about 0.5 wt. %, by weight of composition, of unencapsulated perfume.

### Structurant System

The composition may include a structurant system having at least one structuring agent. The structuring agent may include one or more biopolymers. Non-limiting examples of such biopolymers include polysaccharides such as polymers of glucose, fructose, galactose, mannose, rhamnose, glucuronic acid and mixtures thereof.

The structurant system may be in the form of a polysaccharide system. Preferable polysaccharides include xanthan gum, glucomannan, galactomannan, and combinations thereof. The glucomannan may be derived from a natural gum such as konjac gum. The galactomannan may be derived from natural gums such as locust bean gum and/or tara gum Polysaccharides may also include carrageenan. The polysaccharide(s) may be modified such as by deacetylation.

The composition may include a polysaccharide system comprising at least two polysaccharides, such as a first polysaccharide and a second polysaccharide. The first polysaccharide may be xanthan gum. The second polysaccharide may be selected from the group consisting of glucomannan, galactomannan, and combinations thereof. The second polysaccharide may be selected from the group consisting of konjac gum, locust bean gum, tara gum, and combinations thereof.

Preferably, the first polysaccharide is xanthan gum and the second polysaccharide is konjac gum. The first polysaccharide may be present at a level of greater than 10 wt. % and less than 90 wt. %, alternatively about 20 wt. % to about 80 wt. %, alternatively about 40 wt. % to about 60 wt. %, by weight of the polysaccharide system.

The second polysaccharide may be present at a level of about 15 wt. % to about 85 wt. %, alternatively about 20 wt. % to about 80 wt. %, alternatively about 40 wt. % to about 60 wt. %, by weight of the polysaccharide system.

The total concentration of polysaccharide present in the composition may be less than about 0.5 wt. %, or less than about 0.2 wt. %, or less than about 0.1 wt. %, less than 0.08 wt. %, and less than 0.06 wt. %, specifically reciting all values within these ranges and any ranges created thereby. Without wishing to be bound by theory, it is believed that minimizing the total polysaccharide level present in the composition diminishes residue and/or optimizes spray characteristics.

The polysaccharide system may have a weight-average molecular weight in the range of about 10,000 Daltons to about 15,000,000 Daltons, about 200,000 Daltons to about 10,000,000 Daltons, about 500,000 Daltons to about 9,000,000 Daltons, about 750,000 Daltons to about 8,000,000 Daltons, about 1,000,000 Daltons to about 7,000,000 Daltons, about 2,000,000 Daltons to about 6,000,000 Daltons, about 3,500,000 Daltons to about 6,000,000 Daltons, specifically reciting all values within these ranges and any ranges created thereby.

The polysaccharide may be characterized by the ratio of acetylation. The ratio of acetylation of one or more of the polysaccharides, such as xanthan gum, may be in the range of about 5.0 to about 0.2, in the range of about 3.5 to about 0.3, in the range of about 2.0 to about 0.35, in the range of about 1.5 to about 0.37, in the range of about 1.0 to about 0.39, specifically reciting all values within these ranges and any ranges created thereby.

The composition may have a total protein level of less than about 100 parts per million (ppm), less than 50 ppm, less than 25 ppm, or less than 10 ppm. It may be desirable to limit the total protein level in the composition in order to minimize discoloring of surfaces to which the composition is applied.

### Buffering Agent

The composition may include a buffering agent which may be a carboxylic acid, or a dicarboxylic acid like maleic acid, or a polybasic acid such as citric acid or polyacrylic acid. The acid may be sterically stable, and used in this composition for maintaining the desired pH. The buffering agent may also comprise a base such as triethanolamine, or the salt of an organic acid such as sodium citrate. The composition may have a pH from about 3.0 to about 7.0, about 4.0 to about 6.5, or about 4.0 to about 6.0, specifically reciting all values within these ranges and any ranges created thereby.

### Solubilizer

The composition may contain a solubilizing aid to solubilize any excess hydrophobic organic materials, particularly some malodor counteractants, perfume materials, and also optional ingredients (e.g., insect repelling agent, antioxidant, etc.) which can be added to the composition, that are not readily soluble in the composition, to form a clear, translucent solution. A suitable solubilizing aid is a surfactant, such as a no-foaming or low-foaming surfactant. Suitable surfactants are nonionic surfactants, cationic surfactants, amphoteric surfactants, zwitterionic surfactants, and mixtures thereof.

The composition may contain nonionic surfactants, cationic surfactants, and mixtures thereof. The composition may contain ethoxylated hydrogenated castor oil. One type of suitable hydrogenated castor oil that may be used in the composition is sold as Basophor™, available from BASF.

### Surface Tension Reducing Agent

The composition may include a wetting agent that provides a low surface tension that permits the composition to spread readily and more uniformly on hydrophobic surfaces like polyester and nylon. It has been found that the composition, without such a wetting agent may not spread satisfactorily. The spreading of the composition also allows it to dry faster, so that the treated material is ready to use sooner. Furthermore, a composition containing a wetting agent may penetrate hydrophobic, oily soil better for improved malodor neutralization. A composition containing a wetting agent may also provide improved “in-wear” electrostatic control. For concentrated compositions, the wetting agent facilitates the dispersion of many actives such as antimicrobial actives and perfumes in the concentrated compositions.

Nonlimiting examples of wetting agents include block copolymers of ethylene oxide and propylene oxide. Suitable block polyoxyethylene-polyoxypropylene polymeric surfactants include those based on ethylene glycol, propylene glycol, glycerol, trimethylolpropane and ethylenediamine as the initial reactive hydrogen compound. Polymeric compounds made from a sequential ethoxylation and propoxylation of initial compounds with a single reactive hydrogen atom, such as C₁₂₋₁₈ aliphatic alcohols, are not generally compatible with the cyclodextrin. Certain of the block polymer surfactant compounds include Pluronic® and Tetronic® by the BASF-Wyandotte Corp., Wyandotte, Mich.

Nonlimiting examples of wetting agents of this type are described in U.S. Pat. No. 5,714,137 and include the Silwet surfactants available from Momentive Performance Chemical, Albany, N.Y. Exemplary Silwet surfactants are as follows:

| Name | Average MW |
|---|---|
| L-7608 | 600 |
| L-7607 | 1,000 |
| L-77 | 600 |
| L-7605 | 6,000 |
| L-7604 | 4,000 |
| L-7600 | 4,000 |
| L-7657 | 5,000 |
| L-7602 | 3,000; |

and mixtures thereof.

### Malodor Counteractants

The composition may include other malodor reducing technologies. This may include, without limitation, amine functional polymers, metal ions, cyclodextrins, cyclodextrin derivatives, polyols, oxidizing agents, activated carbon, and combinations thereof.

### Perfume Delivery Technologies

The compositions may comprise one or more perfume delivery technologies that stabilize and enhance the deposition and release of perfume ingredients from treated substrate. Such perfume delivery technologies can also be used to increase the longevity of perfume release from the treated substrate. Perfume delivery technologies, methods of making certain perfume delivery technologies and the uses of such perfume delivery technologies are disclosed in US 2007/0275866 A1.

The compositions may comprise from about 0.001 wt. % to about 20 wt. %, from about 0.01 wt. % to about 10 wt. %, from about 0.05 wt. % to about 5 wt. %, or from about 0.1 wt. % to about 0.5 wt. % by weight of the perfume delivery technology. In one aspect, the perfume delivery technologies may be selected from the group consisting of: pro-perfumes, polymer particles, soluble silicone, polymer assisted delivery, molecule assisted delivery, fiber assisted delivery, amine assisted delivery, cyclodextrins, starch encapsulated accord, zeolite and inorganic carrier, and mixtures thereof.

The perfume delivery technology may comprise an amine reaction product (ARP) or a thio reaction product. One may also use “reactive” polymeric amines and or polymeric thiols in which the amine and/or thiol functionality is pre-reacted with one or more PRMs to form a reaction product. Typically the reactive amines are primary and/or secondary amines, and may be part of a polymer or a monomer (non-polymer). Such ARPs may also be mixed with additional PRMs to provide benefits of polymer-assisted delivery and/or amine-assisted delivery. Nonlimiting examples of polymeric amines include polymers based on polyalkylimines, such as polyethyleneimine (PEI), or polyvinylamine (PVAm). Nonlimiting examples of monomeric (non-polymeric) amines include hydroxyl amines, such as 2-aminoethanol and its alkyl substituted derivatives, and aromatic amines such as anthranilates. The ARPs may be premixed with perfume or added separately in leave-on or rinse-off applications. A material that contains a heteroatom other than nitrogen and/or sulfur, for example oxygen, phosphorus or selenium, may be used as an alternative to amine compounds. The aforementioned alternative compounds can be used in combination with amine compounds. A single molecule may comprise an amine moiety and one or more of the alternative heteroatom moieties, for example, thiols, phosphines and selenols. The benefit may include improved delivery of perfume as well as controlled perfume release. Suitable ARPs as well as methods of making same can be found in USPA 2005/0003980 A1 and U.S. Pat. No. 6,413,920 B1.

### Unencapsulated Perfume

The composition may include unencapsulated perfume comprising one or more perfume raw materials that solely provide a hedonic benefit (i.e. that do not neutralize malodors yet provide a pleasant fragrance). Suitable perfumes are disclosed in U.S. Pat. No. 6,248,135. For example, the composition may include a mixture of volatile aldehydes for neutralizing a malodor and hedonic perfume aldehydes.

Where perfumes, other than the volatile aldehydes in the malodor control component, are formulated into the composition, the total amount of unencapsulated perfumes and volatile aldehydes may be from about 0.015 wt. % to about 3 wt. %, from about 0.01 wt. % to about 1.0 wt. %, from about 0.015 wt. % to about 0.5 wt. %, by weight of the composition.

### Aqueous Carrier

The composition may include an aqueous carrier. The aqueous carrier which is used may be distilled, deionized, or tap water. Water may be present in any amount for the composition to be an aqueous solution. Water may be present in an amount of about 85 wt. % to 99.5 wt. %, about 90 wt. % to about 99.5 wt. %, about 92 wt. % to about 99.5 wt. %, or about 95 wt. %, by weight of the composition. Water containing a small amount of low molecular weight monohydric alcohols, e.g., ethanol, methanol, and isopropanol, or polyols, such as ethylene glycol and propylene glycol, can also be useful. However, the volatile low molecular weight monohydric alcohols such as ethanol and/or isopropanol should be limited since these volatile organic compounds will contribute both to flammability problems and environmental pollution problems. If small amounts of low molecular weight monohydric alcohols are present in the composition due to the addition of these alcohols to such things as perfumes and as stabilizers for some preservatives, the level of monohydric alcohol may about 1 wt. % to about 5 wt. %, alternatively less than about 6 wt. %, alternatively less than about 3 wt. %, alternatively less than about 1 wt. %, by weight of the composition.

### Adjuvants

Adjuvants can be added to the composition herein for their known purposes. Such adjuvants include, but are not limited to, diluents, preservatives, antimicrobial actives, water soluble metallic salts, including zinc salts, copper salts, and mixtures thereof; antistatic agents; insect and moth repelling agents; colorants; antioxidants; aromatherapy agents and mixtures thereof.

### Sprayable Product

The composition may be packaged in a spray dispenser to form a sprayable product. The sprayable product may be suitable for use in air and on surfaces. The spray dispenser includes the nozzle of the present invention.

The sprayable product may be configured to deliver a fine mist. The spray dispenser may be configured in various ways, such as a direct compression-type trigger sprayer, a pre-compression-type trigger sprayer, or an aerosol-type spray dispenser. One suitable spray dispenser is the TS800 Trigger Sprayer (Exxon Mobil PP1063, material classification 10003913, Manufacturer: Calmar).

Another suitable spray dispenser includes a continuous action sprayer, such as FLAIROSOL™ dispenser from Afa Dispensing Group. The FLAIROSOL™ dispenser includes a bag-in-bag or bag-in-can container with a pre-compression spray engine, and aerosol-like pressurization of the composition.

The sprayable product may include a spray engine and a container. The composition may be disposed in the container. The container comprising the composition may be available separately from the spray engine, such a refill container.

### Methods of Use

The composition of the present invention can be used by dispersing, e.g., by placing the aqueous solution into a dispensing means, such as a spray dispenser and spraying an effective amount into the air or onto the desired surface or article. An effective amount as defined herein means an amount sufficient to freshen the air or surface and/or neutralize malodor to the point that it is not discernible by the human sense of smell yet not so much as to saturate or create a pool of liquid on an article or surface and so that, when dry, there is no visual deposit readily discernible. Dispersing can be achieved by using a spray device.

The present disclosure encompasses the method of dispersing an effective amount of the composition onto household surfaces for reducing malodor and/or freshening the household surfaces. The household surfaces are selected from the group consisting of countertops, cabinets, walls, floors, such as carpets or rugs, bathroom surfaces, garbage and/or recycling receptacles, appliances, and kitchen surfaces.

The present disclosure encompasses the method of dispersing a mist of an effective amount of the composition onto fabric and/or fabric articles for reducing malodor and/or freshening the fabric and/or fabric articles. The fabric and/or fabric articles include, but are not limited to, clothes, curtains, drapes, upholstered furniture, carpeting, bed linens, bath linens, tablecloths, sleeping bags, tents, car interior, e.g., car carpet, fabric car seats, shower curtains, etc.

The present disclosure encompasses the method of dispersing a mist of an effective amount of the composition onto and into shoes for reducing malodor impression and/or freshening wherein the shoes are not sprayed to saturation.

The present disclosure relates to the method of dispersing a mist of an effective amount of the composition for into the air for freshening and/or to neutralize malodor.

The present disclosure relates to the method of dispersing a mist of an effective amount of the composition onto cat litter, pet bedding and pet houses for freshening and/or to neutralize malodor.

The present disclosure relates to the method of dispersing a mist of an effective amount of the composition onto household pets for freshening and/or to neutralize malodor.

### Test Methods

### Rheology Test Method

To measure the yield stress and/or the viscosity of a sample, measurements are made with a TA Discovery HR-2 Hybrid Rheometer (TA Instruments, New Castle, Del., U.S.A.) and accompanying TRIOS software version 4.2.1.36612, or equivalent. The instrument is outfitted with a Concentric Cylinder Double Gap Cup (e.g., TA Instrument, cat. #546050.901), Double Gap Rotor (e.g. TA Instruments, cat. #546049.901) and Split Cover (e.g. TA Instruments, cat. #545626.001). The calibration is done in accordance with manufacturer recommendations. A refrigerated, circulating water bath set to 25° C. is attached to the Concentric Cylinder. The Concentric Cylinder temperature is set to 25° C. The temperature is monitored within the Control Panel until the instrument reaches the set temperature, then an additional 5 minutes is allowed to elapse to ensure equilibration before loading sample material into the Double Gap Cup.

The parameters for the Double Gap Cup are as follows: the inside cup diameter is 30.2 mm; the inside bob diameter is 32 mm; the outside bob diameter is 35 mm; the outside cup diameter is 37 mm; the inner cylinder height is 55 mm; the immersed height is 53 mm; the operating gap is 2,000.0 μm; the loading gap is 90,000.0 μm; the Environmental system is Peltier; and with a sample volume between 12 ml and 15 ml (preferably 12 ml). The Concentric Cylinder Double Gap Cup is covered with a solvent trap to prevent drying during testing (e.g. 545626.001, Split Cover for DHR & AR-Series Smart Swap Peltier Concentric Cylinders).

To load a new sample, the Double Gap Cup is cleaned, dried and re-assembled in accordance with the manufacturer's instructions. A minimum of 12 ml of sample is added to the Double Gap Cup using a syringe, and the sample is then allowed to sit for 15 minutes, ensuring that any trapped air bubbles rise to the surface. The Double Gap Rotor is then lowered to the proper gap and data are collected in accordance with the following Settings and Procedures in a series of steps conducted in the following order:

### Load a New Sample.

The first Conditioning Sample Step is conducted using the following instrumental settings: Environmental Control is set with a Temperature of 25° C.; Inherit Set Point is selected as Off; Soak Time is set to 0.0 s; Wait for Temperature is selected as On; Wait for axial force is selected as Off; Preshear Options is set with a Perform Preshear selected as On, with the shear rate set at 100 s⁻¹, and the Duration set as 60.0 s; Equilibrium is set with a Perform Equilibration selected as On; and Duration is set to 1,800.0 s.

The Flow Peak Hold Step is conducted using the following instrument settings: Environmental Control is set with a Temperature of 25° C.; Inherit Set Point is selected as Off; Soak Time is set to 0.0 s; Wait for Temperature is selected as Off; Test Parameters is set with a Duration of 3,000.0 s; Shear Rate is selected and set to 0.01 s-1; Inherit initial value is selected as Off; Sampling interval is selected and set to 3.0 s/pt.; Controlled Rate Advanced is set with a Motor mode selected as Auto; Data acquisition is set with a End of step selected as Zero torque; Fast sampling is selected as Off; Save image is selected as Off; Step Termination is set with Limit checking Enabled selected as Off Equilibrium Enabled is selected as Off; Step Repeat Enabled is selected as Off.

The Yield Stress is calculated from the data collected in the Flow Peak Hold Step, in the following way: The data points are plotted as Stress (mPa) on the y-axis against Percent Stain (%) on the x-axis. The Yield Stress is determined by selecting the “Analysis” tab, then selecting “Signal max” from the Function drop down list and finally selecting “Analyze” in the Commands category. For a contiguous data set (containing a single stress value greater than zero for each time value), the Yield Stress equals the value of ‘Max Y’ if it occurs in the first 2,500% strain, and proceed to second Conditioning Sample Step and Flow Sweep Step.

If, however, the ‘Max Y’ occurs after the first 2,500% strain when the Shear Rate was set at 0.01 s-1, load a new sample and repeat the first Conditioning Sample Step and the Flow Peak Hold Step, after setting Shear Rate in the Flow Peak Hold Step to 0.03 s-1. Repeat the Yield Stress calculation. Yield Stress equals the value of ‘Max Y’ if it occurs in the first 2,500% strain, and proceed to second Conditioning Sample Step and Flow Sweep Step.

If, however, the ‘Max Y’ occurs after the first 2,500% strain when the Shear Rate was set at 0.03 s-1, load a new sample and repeat the first Conditioning Sample Step and the Flow Peak Hold Step, after setting Shear Rate in the Flow Peak Hold Step to 0.10 s-1. Repeat the Yield Stress calculation. Yield Stress equals the value of ‘Max Y’ if it occurs in the first 2,500% strain, and proceed to second Conditioning Sample Step and Flow Sweep Step.

If, however, the ‘Max Y’ occurs after the first 2,500% strain when the Shear Rate was set at 0.10 s-1, load a new sample and repeat the first Conditioning Sample Step and the Flow Peak Hold Step, after setting Shear Rate in the Flow Peak Hold Step to 0.30 s-1. Repeat the Yield Stress calculation. Yield Stress equals the value of ‘Max Y’ if it occurs in the first 2,500% strain, and proceed to second Conditioning Sample Step and Flow Sweep Step.

If, however, the ‘Max Y’ occurs after the first 2,500% strain when the Shear Rate was set at 0.30 s-1, load a new sample and repeat the first Conditioning Sample Step and the Flow Peak Hold Step, after setting Shear Rate in the Flow Peak Hold Step to 1.00 s-1. Repeat the Yield Stress calculation. Yield Stress equals the value of ‘Max Y’ if it occurs in the first 2,500% strain; if there is there is ‘Max Y’ value or if the ‘Max Y’ occurs after the first 2,500% strain, the Yield Stress equals zero.

### Load a New Sample.

The second Conditioning Sample Step is conducted using the following instrumental settings: Environmental Control is set with a Temperature of 25° C.; Inherit Set Point is selected as Off; Soak Time is set to 10.0 s; Wait for Temperature is selected as Off; Wait for axial force is selected as Off; Preshear Options is set with a Perform Preshear selected as Off; Equilibrium is set with a Perform Equilibration selected as On; and Duration is set to 1,800.0 s.

The Flow Sweep Step is conducted using the following instrument settings: Environmental Control is set with a Temperature of 25° C.; Inherit Set Point is selected as Off; Soak Time is set to 0.0 s; Wait For Temperature is selected as Off; Test Parameters is set with Logarithmic sweep selected; Shear rate is selected and set to 1.0e-3 s-1 to 1000.0 s-1; Points per decade is set to 5; Steady state sensing is selected as On; Max equilibration time is set to 45.0 s; Sample period is set to 5.0 s; % tolerance is set to 5.0; Consecutive within is set to 3; Scaled time average is selected as Off; Controlled Rate Advanced is set with Motor mode selected as Auto; Data acquisition is set with Save point display selected as Off; Save image is selected as Off; Step termination is set with Limit checking Enabled selected as Off; Equilibrium Enabled is selected as Off, Step Repeat Enabled is selected as Off.

The Viscosity is calculated from the data collected in the Flow Sweep Test, and determined to be the ‘infinite rate viscosity’ determined by selecting the ‘Best Fit Flow (viscosity vs. rate)’ for the viscosity curve in the analysis portion of the program, expressed in mPa·s.

### Spray Rate Test Method

The composition is contained in an airtight pressure pot, pressurized with a nitrogen head space where the pressure is maintained with a pressure regulator. When the flow control valve is open, the composition flow through a fixture holding the manifold and nozzle of the trigger sprayer for 10 seconds directly into a beaker. The Spray Rate is the number of grams collected per second in the beaker. The manifold and nozzle can be changed to accommodate the multiple nozzle designs (Table 6).

### Spray D(90) Normalized and Spray D(4,3) Normalized Test Method

Spray droplet volume size distribution measurements comprising Spray D(90) Normalized and Spray D(4,3) Normalized values were determined using a Malvern Spraytec 2000 laser diffraction spray droplet sizing instrument (supplied by Malvern Instruments, Worcestershire, UK), equipped with a 300 mm lens possessing a focal length of the 150 mm, and an Air Purge System (not greater than 14.5 psi). The system was controlled with a computer and software accompanying the instrument, such as the Spraytec software version 3.20 or equivalent, utilizing Mie Theory and Fraunhofer Approximation optical theory. The system was placed in a fume hood for atmospheric control with care taken to place it directly opposite the actuation spray plume trajectory to prevent saturation, with an air flow rate of between 50-70 L/min (60 L/min was the target rate). The distance from the dispensing nozzle orifice to the laser during measurements was 15 cm. During spray droplet analysis, each sample was dispensed from a pressurized system set to constant pressure. A new trigger sprayer and new nozzle was used for each sample replicate analyzed. Lighting conditions were not changed during or between the background control and test sample data collection periods. Light obscuration values below 95% were considered suitable to provide accurate results.

Spray measurements were conducted using the following spray SOP instrument configuration: Rapid SOP type was chosen, and the following settings were selected: Hardware Configuration set to “Default”, Measurement Type set to “Rapid”, Data Acquisition Rate set to “250 Hz”, and Lens Type set to “300”. Within the Measurement menu: Background set to “2 seconds”, Inspection was selected, the box under Output Trigger was Unchecked. Under the Measurement tab “Rapid” was selected, Events Number set to “1”, Duration Per Event set to “4000.0”, Units set to “ms”. Measurement Trigger where Trigger Type set to “Transmission drops to level” and Transmission set to “96”, Data Collection where Start is set to “0.0”, Units set to “ms”, and select “before the trigger” from the drop down menu. On the Advanced tab window all boxes were Unchecked, and Grouping was “no grouping”; The Background Alarms was set to “default values”. On the Analysis Tab and under Optical Properties, Particle Set was set to “Water”, Dispersant set to “Air”, Multiple Scattering Analysis set to “Enable”. On the Data Handling tab and under Detector Range was set to “first-8 and last”, “No extinction analysis” box was selected, Scattering threshold was set to “1”. On the Data Handling/Spray Profile the Path Length was set to “100.0”, the Alarm was selected, and the “Use default values” box was checked. On the Additional Properties tab the Curve Fit was set to “no fit”, User Size was set to “enable box”, the drop down menu as set to “Default”. On the Additional Properties/Advanced tab Particle Diameter was set to “0.10” for the minimum and to “900” for the maximum, and Result Type was set to “Volume Distribution”. On the Output tab, Export Option was set to “not selected”, the Derived Parameter was selected, the Use Averaging Period box was selected and set to “0.0” and “ms”. On the Average menu “Average scatter data” was selected. Spray measurements were conducted using the following Spray Procedure: The sample was first test sprayed from the sprayer for 1-2 seconds, to ensure that the nozzle was free flowing and not clogged; the sample was loaded into the holding device in the front of the Spraytec 2000 system. The sprayer was sprayed at constant pressure for 6 seconds The spray droplet size data were viewed and saved as “Average Scatter Data”.

All measurements were conducting using the instrument configuration procedures specified and, using great care to ensure that the pressure applied was the same for all samples.

D(90) and D(4,3) were values obtained from the instrument software for both the Example and Control samples separately.

Each of the Spray D(90) Normalized, D(4,3) Normalized, D(10) Normalized, D(50) Normalized, and D(3,2) Normalized values reported for each of the samples in the Example Table 5 is the average value calculated from five replicate spray plumes per sample.

### Spray Pattern Test Method

The sprayer is affixed in position 15 cm away from a piece of high-contrast pH paper measuring 20 cm×20 cm. The pH paper is held flat and is positioned such that the plane created by the large surface of the paper is aligned normal the bench top (parallel to gravity). The output of the sprayer is oriented such that the initial output from the nozzle is parallel to the bench (normal to gravity) and is aligned directly at the center of the pH paper. The composition is sprayed with constant pressure of either 72 psi (Table 7) or 94 psi (Table 8) for precisely 0.5 seconds, which leaves a pattern on the pH paper. The radius of the pattern on the pH paper is the longest measured using a line gauge, by measuring the distance from the center of the pH paper to the edge of the pattern while circumventing the entire pattern. The diameter of the spray pattern is calculated as twice the radius, and reported in mm.

### Examples

TABLE 1
Materials

| | Ingredient | Manufacturer | Lot Number |
|---|---|---|---|
| 1 | Deionized Water | — | — |
| 2 | Koralone B-119 | Rohm & Haas/DOW | YY00I5H902 |
| 3 | Ethanol | Grain Processing Corp | 1B0002639378 |
| 4 | Diethylene Glycol | OLD WORLD | MP01K14002 |
| | | INDUSTRIES INC | |
| 5 | Hydroxypropyl Beta | WACKER CEHMICAL | 74LE401 |
| | CD | CORP | |
| 6 | Citric Acid | Archer Daniels Midland | S908061 |
| 7 | Konjac gum- Newstar | Sichuan Newstar Konjac | 2020031021 |
| | SLT1420 | Co., Ltd | |
| 8 | Xanthan gum CP | CPKelco | 0C0018K |
| | Kelco, Keltrol CC | | |
| 9 | Voyager Zen PAC PC | Encapsys | 202017206 |
| 10 | Silwet L-700 | MPM | 20GSVU165 |
| 11 | Sodium Chloride | Morton Salt, Inc. | RI19249043 |

### Preparation of Aqueous Pre-Mix (Solution 1)

An aqueous premix was prepared that contained water and salts is prepared in the 1,000 L mix tank. The water (1) was added to the mix tank in amount indicated in Table 2. A stirrer was used to add vigorous agitation in the water. The remaining ingredients were added in amounts indicated in Table 2, and stirring continued until all the materials were completely dissolved.

TABLE 2
Composition of Aqueous Premix

| Ingredient | Mass added (g) |
|---|---|
| Water (1) | 93,6200 |
| Koralone B-119 (2) | 828 |
| Ethanol (3) | 34,000 |
| Diethylene Glycol (4) | 1,064 |
| Hydroxypropyl Beta CD (5) | 7,889 |
| Citric Acid (6) | 542 |

### Preparation of 1 wt % Xanthan Gum Stock Solution (Solution 2)

A xanthan gum stock solution was prepared to ensure hydration of the gum prior to further making. 40,000 grams of a 1 wt % xanthan gum solution are prepared using the Quadro CC0 high sheer mixer with powder feeder. Dry xanthan gum powdered (8) is pushed through the feeder with a correct amount of water (1), to create a 1 wt % solutions. They mixture is allowed to equilibrate without agitation for 24 hours, to ensure complete hydration of the gum before using.

### Preparation of 1 wt % Konjac Gum Stock Solution (Solution 3)

A konjac gum stock solution was prepared to ensure hydration of the gum prior to further making. 40,000 grams of a 1 wt % konjac gum solution are prepared using the Quadro CC0 high sheer mixer with powder feeder. Dry konjac gum powdered (7) is pushed through the feeder with a correct amount of water (1), to create a 1 wt % solutions. The mixture is allowed to equilibrate without agitation for 24 hours, to ensure complete hydration of the gum before using.

### Preparation of Final Composition (Solution 4)

The preparation of the final composition was achieved by adding both gum pre-mixes and all the final ingredients to the mix tank. For the first addition, 31,400 grams of konjac gum pre-mix (Solution 3) was added to the mix tank with the composition previously described (Solution 1), and thoroughly mixed. For the next addition, 21,000 grams of xanthan gum pre-mix (Solution 2) was added to this mix tank and thoroughly mixed. The composition is continually mixed until the measurement of yield stress greater than zero, by the RHEOLOGY TEST METHOD. For the final addition, the remaining ingredients are added in amounts described in Table 3, with sufficient mixing to reduce the particle size of the perfume consistent with the specification.

TABLE 3
Final Additions to the Composition

| Ingredient | Mass added (g) |
|---|---|
| Voyager PAC PMC (9) | 10,700 |
| Silwet L7600 (10) | 1,051 |
| Sodium Chloride (11) | 1,057 |
| Neat Perfume/solubilizer | 4,416 |

The final composition (Solution 4) is loaded into the pressure pot for spray measurements. The spray assembly was outfitted with a nozzle (Table 6). The pressure pot was pressured with nitrogen to either 72 psi or 94 psi. The drop size distribution of the spray at each pressure and each nozzle was measured by the SPRAY D(90) NORMALIZED AND SPRAY D(4,3) NORMALIZED TEST METHOD. The spray rate at each pressure and each nozzle was measured by the SPRAY RATE TEST METHOD. The spray pattern at each pressure and each nozzle was measured by the SPRAY PATTERN TEST METHOD. These results are tabled for each pressure in Table 5 and Table 6.

TABLE 4
Dimensions of Tested Nozzles

| | | | Ratio of Outlet |
|---|---|---|---|
| | Outlet Orifice | Micro Orifice | Orifice Diameter |
| Nozzle | Axial Length | Diameter | to Outlet Orifice |
| ID | (mm) | (mm) | Axial Length |
| 2a | 0.48 | 0.79 | 1.64 |
| 2b | 0.48 | 0.80 | 1.66 |
| 4a | 0.63 | 0.60 | 0.96 |
| 4b | 0.63 | 0.59 | 0.94 |
| 4c | 0.63 | 0.60 | 0.96 |
| 7a | 0.48 | 0.60 | 1.24 |
| 7b | 0.48 | 0.59 | 1.22 |
| 7c | 0.48 | 0.60 | 1.23 |
| 9a | 0.48 | 0.58 | 1.21 |
| 9b | 0.48 | 0.59 | 1.22 |
| 9c | 0.48 | 0.60 | 1.23 |
| 1a | 0.48 | 0.42 | 0.86 |
| 1b | 0.48 | 0.41 | 0.85 |
| 1c | 0.48 | 0.42 | 0.86 |
| 3a | 0.34 | 0.58 | 1.70 |
| 3b | 0.34 | 0.57 | 1.69 |
| 5a | 0.48 | 0.44 | 0.92 |
| 5b | 0.48 | 0.41 | 0.85 |
| 5c | 0.48 | 0.44 | 0.91 |
| 6a | 0.48 | 0.56 | 1.16 |
| 6b | 0.48 | 0.53 | 1.09 |
| 6c | 0.48 | 0.53 | 1.09 |
| 8a | 0.48 | 0.55 | 1.15 |
| 8b | 0.48 | 0.55 | 1.15 |
| 8c | 0.48 | 0.58 | 1.19 |
| 10a | 0.48 | 0.53 | 1.10 |
| 10b | 0.48 | 0.53 | 1.10 |
| 10c | 0.48 | 0.53 | 1.10 |
| 12a | 0.18 | 0.58 | 3.14 |
| 12b | 0.18 | 0.58 | 3.14 |
| 12c | 0.18 | 0.58 | 3.15 |

TABLE 5
Low Pressure (72 psi) Spray Characteristics

| | Ratio of | | | | | | | ph Paper |
|---|---|---|---|---|---|---|---|---|
| | Outlet Orifice | | | | | | | Spray |
| | Diameter to | | | | | | Spray | Pattern |
| Nozzle | Outlet Orifice | D(10) | D(50) | D(90) | D(3, 2) | D(4, 3) | Rate | Diameter |
| ID | Axial Length | (um) | (um) | (um) | (um) | (um) | (g/s) | (mm) |
| 2a | 1.64 | 89.93 | 264.46 | 584.13 | 185.57 | 305.56 | 5.66 | 140 |
| 2b | 1.66 | 67.38 | 187.25 | 449.60 | 134.56 | 228.76 | 4.99 | 146 |
| 4a | 0.96 | 124.72 | 323.46 | 643.53 | 227.17 | 357.45 | 6.44 | 76 |
| 4b | 0.94 | 73.05 | 202.41 | 447.04 | 148.38 | 236.67 | 4.34 | 127 |
| 4c | 0.96 | 88.21 | 251.13 | 544.91 | 177.71 | 289.13 | 5.35 | 76 |
| 7a | 1.24 | 124.72 | 323.46 | 643.53 | 227.17 | 357.45 | 5.75 | 89 |
| 7b | 1.22 | 73.99 | 218.21 | 500.28 | 153.16 | 258.18 | 5.56 | 146 |
| 7c | 1.23 | 111.88 | 284.92 | 582.82 | 207.08 | 320.16 | 5.75 | 102 |
| 9a | 1.21 | 124.72 | 323.46 | 643.53 | 227.17 | 357.45 | 6.08 | 76 |
| 9b | 1.22 | 261.04 | 478.41 | 771.09 | 387.38 | 496.35 | 6.38 | 76 |
| 9c | 1.23 | 98.23 | 314.67 | 642.58 | 192.07 | 346.99 | 6.3 | 121 |
| 1a | 0.86 | 51.04 | 118.08 | 271.76 | 92.09 | 144.35 | 2.78 | 76 |
| 1b | 0.85 | 62.07 | 180.52 | 439.48 | 130.53 | 221.10 | 2.62 | 89 |
| 1c | 0.86 | 124.72 | 323.46 | 643.53 | 227.17 | 357.45 | 2.78 | 102 |
| 3a | 1.70 | 47.35 | 107.65 | 335.43 | 92.02 | 156.05 | 4.11 | 140 |
| 3b | 1.69 | 57.12 | 129.51 | 311.36 | 103.35 | 161.99 | 4.98 | 127 |
| 5a | 0.92 | 59.36 | 171.48 | 381.76 | 121.68 | 200.69 | 3.3 | 102 |
| 5b | 0.85 | 50.14 | 113.45 | 251.79 | 90.91 | 135.34 | 2.6 | 102 |
| 5c | 0.91 | 56.10 | 169.56 | 433.05 | 114.06 | 212.77 | 3.16 | 51 |
| 6a | 1.16 | 124.72 | 323.46 | 643.53 | 227.17 | 357.45 | 4.26 | 102 |
| 6b | 1.09 | 60.93 | 156.91 | 359.92 | 119.90 | 188.29 | 3.88 | 76 |
| 6c | 1.09 | 57.09 | 175.33 | 409.74 | 121.33 | 209.56 | 3.96 | 114 |
| 8a | 1.15 | 69.87 | 245.92 | 551.51 | 148.14 | 283.69 | 4.3 | 89 |
| 8b | 1.15 | 124.72 | 323.46 | 643.53 | 227.17 | 357.45 | 4.2 | 76 |
| 8c | 1.19 | 88.96 | 280.76 | 597.98 | 180.52 | 317.15 | 4.5 | 127 |
| 10a | 1.10 | 124.72 | 323.46 | 643.53 | 227.17 | 357.45 | 4.01 | 114 |
| 10b | 1.10 | 79.09 | 266.96 | 581.27 | 171.94 | 303.58 | 4.36 | 76 |
| 10c | 1.10 | 65.85 | 193.15 | 446.29 | 136.26 | 230.29 | 4.1 | 140 |
| 12a | 3.14 | 53.63 | 141.74 | 360.59 | 105.20 | 179.94 | 4.88 | 171 |
| 12b | 3.14 | 61.70 | 134.67 | 305.39 | 114.14 | 163.29 | 4.77 | 165 |
| 12c | 3.15 | 56.14 | 163.56 | 397.81 | 107.36 | 200.88 | 5.39 | 127 |

TABLE 6
Medium Pressure (94 psi) Spray Characteristics

| | Ratio of | | | | | | | pH Paper |
|---|---|---|---|---|---|---|---|---|
| | Outlet Orifice | | | | | | | Spray |
| | Diameter to | | | | | | Spray | Pattern |
| Nozzle | Outlet Orifice | Dv10 | Dv50 | Dv90 | D[3][2] | D[4][3] | Rate | Diameter |
| ID | Axial Length | (um) | (um) | (um) | (um) | (um) | (g/s) | (mm) |
| 2a | 1.64 | 75.45 | 232.36 | 553.62 | 161.41 | 278.49 | 6.07 | 152 |
| 2b | 1.66 | 58.23 | 177.16 | 476.32 | 117.2 | 228.34 | 6.52 | 165 |
| 4a | 0.96 | 124.72 | 323.46 | 643.53 | 227.17 | 357.45 | 6.52 | 89 |
| 4b | 0.94 | 50.19 | 147.04 | 357.23 | 92.83 | 180.25 | 4.72 | 140 |
| 4c | 0.96 | 80.82 | 251.86 | 560.45 | 156.88 | 291.44 | 6.52 | 102 |
| 7a | 1.24 | 124.72 | 323.46 | 643.53 | 227.17 | 357.45 | 6.7 | 114 |
| 7b | 1.22 | 64.57 | 198.21 | 492.7 | 133.18 | 244.06 | 6.02 | 146 |
| 7c | 1.23 | 88.19 | 257.32 | 569.75 | 174.18 | 298.18 | 6.32 | 140 |
| 9a | 1.21 | 124.72 | 323.46 | 643.53 | 227.17 | 357.45 | 6.97 | 76 |
| 9b | 1.22 | 200.94 | 419.73 | 734.56 | 311.95 | 443.78 | 7.44 | 89 |
| 9c | 1.23 | 123.45 | 356.3 | 690.01 | 229.73 | 385.1 | 7.25 | 121 |
| 1a | 0.86 | 63.14 | 170.9 | 390.57 | 122.04 | 204.13 | 2.91 | 89 |
| 1b | 0.85 | 53.36 | 133.32 | 343.56 | 103.92 | 171.69 | 2.93 | 102 |
| 1c | 0.86 | 124.72 | 323.46 | 643.53 | 227.17 | 357.45 | 3.1 | 102 |
| 3a | 1.70 | 43.21 | 86.98 | 252.86 | 78.47 | 123.44 | 4.8 | 152 |
| 3b | 1.69 | 48.92 | 98.53 | 218.4 | 84.82 | 120.25 | 5.67 | 140 |
| 5a | 0.92 | 47.22 | 128.98 | 310.06 | 89.25 | 157.93 | 3.78 | 121 |
| 5b | 0.85 | 43.99 | 90.75 | 195.18 | 72.91 | 108.21 | 2.65 | 114 |
| 5c | 0.91 | 44.18 | 96.98 | 214.56 | 76.64 | 116.26 | 3.44 | 51 |
| 6a | 1.16 | 124.72 | 323.46 | 643.53 | 227.17 | 357.45 | 4.75 | 102 |
| 6b | 1.09 | 57.76 | 175.37 | 441.78 | 122.4 | 217.17 | 4.66 | 102 |
| 6c | 1.09 | 53.74 | 163.06 | 411.57 | 108.42 | 203.74 | 4.71 | 127 |
| 8a | 1.15 | 113.41 | 311.55 | 633.17 | 211.2 | 346.46 | 5.01 | 102 |
| 8b | 1.15 | 124.72 | 323.46 | 643.53 | 227.17 | 357.45 | 5.06 | 102 |
| 8c | 1.19 | 60.18 | 208.18 | 498.59 | 125.35 | 249.35 | 4.83 | 127 |
| 10a | 1.10 | 124.72 | 323.46 | 643.53 | 227.17 | 357.45 | 4.88 | 127 |
| 10b | 1.10 | 52.88 | 153.32 | 384.22 | 102.7 | 191.44 | 4.67 | 102 |
| 10c | 1.10 | 51.78 | 142.09 | 376.37 | 103.55 | 184.28 | 4.76 | 140 |
| 12a | 3.14 | 50.9 | 133.32 | 352.77 | 98.65 | 173.5 | 5.9 | 197 |
| 12b | 3.14 | 54.18 | 104.2 | 242.91 | 94.43 | 131.31 | 5.13 | 152 |
| 12c | 3.15 | 73.49 | 232.77 | 523.87 | 154.80 | 271.02 | 6.31 | 127 |

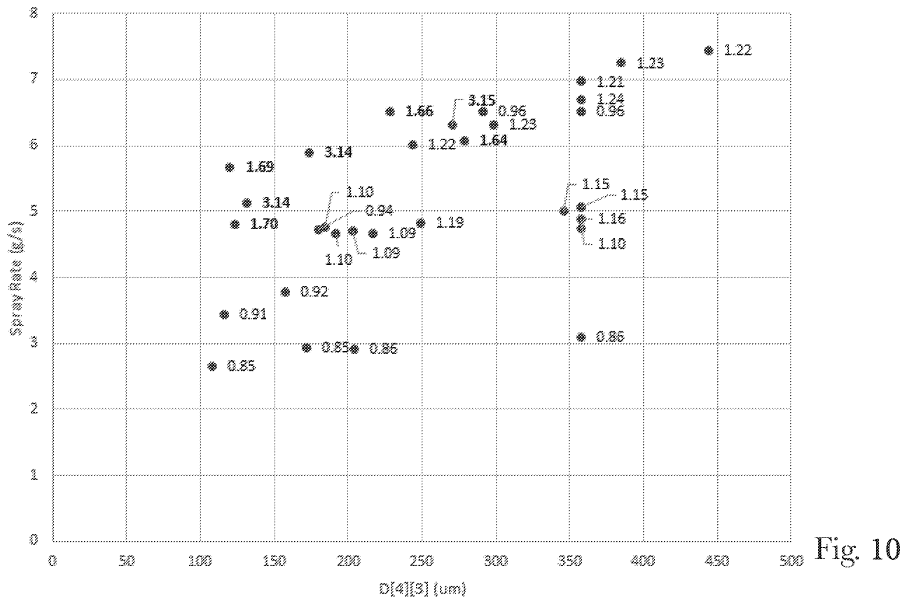

FIG. 10 is a plot of the results provided in Table 6, with the text adjacent to the data points represents the value of the ratio of the outlet orifice diameter to the axial length of the nozzle. As shown in FIG. 10, a combination of relatively high spray rates and relatively low D[4] [3] droplet sizes correlate with a ratio of the outlet orifice diameter to the axial length from about 1.3 to about 3.5.

### “Combinations:”

A. Spray product comprising a composition contained within a reservoir, wherein the composition has a yield stress greater than zero and less than 1,000 mPa as determined by the RHEOLOGY TEST METHOD, wherein the spray product further comprises:
a valve in composition communication with the reservoir;
an actuator in mechanical communication with the valve;
a nozzle having an outlet orifice;
a swirl chamber in composition communication with the outlet orifice; and
a plurality of swirl chamber swirl chamber inlet channels in composition communication with the swirl chamber,
wherein the outlet orifice is defined by an outlet orifice diameter and an outlet orifice axial length, wherein a ratio of the outlet orifice diameter to the axial length is from about 1.3 to about 3.5.
   B. The spray product of Paragraph A, wherein the composition has a viscosity in the range of about 1 mPa-s to about 20 mPa-s, preferably about 1 mPa-s to about 15 mPa-s, more preferably about 1 mPa-s to about 10 mPa-s, or most preferably about 1 mPa-s to about 5 mPa-s,
   C. The spray product of Paragraph A or Paragraph B, wherein the ratio of the outlet orifice diameter to the outlet orifice axial length is from about 1.5 to about 3.2.
   D. The spray product of any of Paragraphs A through C, wherein the composition comprises a plurality of particles and a structurant system.
   E. The spray product of any of Paragraphs A through D, wherein the structurant system comprises xanthan gum and a polysaccharide selected from the group consisting of: konjac gum, locust bean gum, tara gum, and combinations of.
   F. The spray product of any of Paragraphs A through E, wherein the sprayer comprises a pre-compression pump assembly.
   G. Spray product comprising a composition contained within a reservoir, wherein the composition comprises a plurality of particles and a structurant system comprising xanthan gum a polysaccharide selected from the group consisting of: konjac gum, locust bean gum, tara gum, and combinations thereof, and wherein the composition comprises a plurality of particles, wherein the spray product further comprises:
a valve in composition communication with the reservoir;
an actuator in mechanical communication with the valve;
a nozzle having an outlet orifice;
a swirl chamber in composition communication with the outlet orifice; and
a plurality of swirl chamber swirl chamber inlet channels in composition communication with the swirl chamber,
wherein the outlet orifice is defined by an outlet orifice diameter and an outlet orifice axial length, wherein a ratio of the outlet orifice diameter to the axial length is from about 1.3 to about 3.5.
   H. The spray product of Paragraph G, wherein the composition has a viscosity in the range of 1 mPa-s to about 20 mPa-s, preferably about 1 mPa-s to about 15 mPa-s, more preferably about 1 mPa-s to about 10 mPa-s, or most preferably about 1 mPa-s to about 5 mPa-s.
   I. The spray product of Paragraph G or Paragraph H, wherein the ratio of the outlet orifice diameter to the outlet orifice axial length is from about 1.5 to about 3.2.
   J. The spray product of any of Paragraphs G through I, wherein the sprayer comprises a pre-compression pump assembly.
   K. A method of freshening the air, the method comprising the steps of:
providing a sprayer comprising:
   a valve in composition communication with the reservoir;
   an actuator in mechanical communication with the valve;
   a nozzle having an outlet orifice;
   a swirl chamber in composition communication with the outlet orifice; and
   a plurality of swirl chamber swirl chamber inlet channels in composition communication with the swirl chamber,
   wherein the outlet orifice is defined by an outlet orifice diameter and an outlet orifice axial length, wherein a ratio of the outlet orifice diameter to the axial length is from about 1.3 to about 3.5; and
spraying a composition from the sprayer, wherein the composition comprises a plurality of particles and a structurant system comprising xanthan gum a polysaccharide selected from the group consisting of: konjac gum, locust bean gum, tara gum, and combinations thereof, and wherein the composition comprises a plurality of particles
   L. The method of Paragraph K, wherein the composition has a viscosity in the range of 1 mPa-s to about 20 mPa-s, preferably about 1 mPa-s to about 15 mPa-s, more preferably about 1 mPa-s to about 10 mPa-s, or most preferably about 1 mPa-s to about 5 mPa-s.
   M. The method of Paragraph K or Paragraph L wherein the ratio of the outlet orifice diameter to the outlet orifice axial length is from about 1.5 to about 3.2.
   N. The method of Paragraph M, wherein the sprayer comprises a pre-compression pump assembly.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A spray product comprising a composition contained within a reservoir, wherein the composition has a yield stress greater than zero and less than 1,000 mPa as determined by the RHEOLOGY TEST METHOD, wherein the spray product further comprises:
a valve in composition communication with the reservoir;
an actuator in mechanical communication with the valve;
a nozzle having an outlet orifice;
a swirl chamber in composition communication with the outlet orifice; and
a plurality of swirl chamber swirl chamber inlet channels in composition communication with the swirl chamber,
wherein the outlet orifice is defined by an outlet orifice diameter and an outlet orifice axial length, wherein a ratio of the outlet orifice diameter to the axial length is from about 1.3 to about 3.5.

2. The spray product of claim 1, wherein the composition has a viscosity in the range of about 1 mPa-s to about 20 mPa-s.

3. The spray product of claim 1, wherein the ratio of the outlet orifice diameter to the outlet orifice axial length is from about 1.5 to about 3.2.

4. The spray product of claim 1, wherein the composition comprises a plurality of particles and a structurant system.

5. The spray product of claim 1, wherein the structurant system comprises xanthan gum and a polysaccharide selected from the group consisting of: konjac gum, locust bean gum, tara gum, and combinations of.

6. The spray product of claim 1, wherein the sprayer comprises a pre-compression pump assembly.

7. The spray product of claim 1, wherein the composition has a viscosity in the range of about 1 mPa-s to about 15 mPa-s.

8. The spray product of claim 1, wherein the composition has a viscosity in the range of about 1 mPa-s to about 10 mPa-s.

9. The spray product of claim 1, wherein the composition has a viscosity in the range of about or most preferably about 1 mPa-s to about 5 mPa-s.

10. A spray product comprising a composition contained within a reservoir, wherein the composition comprises a plurality of particles and a structurant system comprising xanthan gum a polysaccharide selected from the group consisting of: konjac gum, locust bean gum, tara gum, and combinations thereof, and wherein the composition comprises a plurality of particles, wherein the spray product further comprises:
a valve in composition communication with the reservoir;
an actuator in mechanical communication with the valve;
a nozzle having an outlet orifice;
a swirl chamber in composition communication with the outlet orifice; and
a plurality of swirl chamber swirl chamber inlet channels in composition communication with the swirl chamber,
wherein the outlet orifice is defined by an outlet orifice diameter and an outlet orifice axial length, wherein a ratio of the outlet orifice diameter to the axial length is from about 1.3 to about 3.5.

11. The spray product of claim 10, wherein the composition has a viscosity in the range of 1 mPa-s to about 20 mPa-s.

12. The spray product of claim 10, wherein the ratio of the outlet orifice diameter to the outlet orifice axial length is from about 1.5 to about 3.2.

13. The spray product of claim 10, wherein the sprayer comprises a pre-compression pump assembly.

14. A method of freshening the air, the method comprising the steps of:
providing a sprayer comprising:
a valve in composition communication with the reservoir;
an actuator in mechanical communication with the valve;
a nozzle having an outlet orifice;
a swirl chamber in composition communication with the outlet orifice; and
a plurality of swirl chamber swirl chamber inlet channels in composition communication with the swirl chamber,
wherein the outlet orifice is defined by an outlet orifice diameter and an outlet orifice axial length, wherein a ratio of the outlet orifice diameter to the axial length is from about 1.3 to about 3.5; and
spraying a composition from the sprayer, wherein the composition comprises a plurality of particles and a structurant system comprising xanthan gum a polysaccharide selected from the group consisting of: konjac gum, locust bean gum, tara gum, and combinations thereof, and wherein the composition comprises a plurality of particles

15. The method of claim 14, wherein the composition has a viscosity in the range of 1 mPa-s to about 20 mPa-s.

16. The method of claim 14, wherein the ratio of the outlet orifice diameter to the outlet orifice axial length is from about 1.5 to about 3.2.

17. The method of claim 14, wherein the sprayer comprises a pre-compression pump assembly.
